(19) United States
(12) Patent Application Publication
(10) Pub. No.: US 2019/0134351 A1
(43) Pub. Date: May 9, 2019

(54) GARMENT WITH DIALYSIS CATHETER ACCESS AND DIALYSIS TUBE MANAGEMENT SYSTEM

(71) Applicant: Steven J. Poutre, Macedon, NY (US)

(72) Inventor: Steven J. Poutre, Macedon, NY (US)

(21) Appl. No.: 16/112,686

(22) Filed: Aug. 25, 2018

Related U.S. Application Data
(60) Provisional application No. 62/553,277, filed on Sep. 1, 2017.

(51) Int. Cl. A61M 25/02; A61M 1/14; A61M 1/36; A41D 27/28; A41D 27/10

(52) U.S. Cl. CPC A61M 25/02; A61M 1/14; A61M 1/3661; A41D 27/285; A41D 2400/32; A61M 2025/0206; A41D 2300/326; A41D 2300/322; A41D 2300/324; A41D 27/10

(57) ABSTRACT

A garment includes a right side chest area multi-directional opening and a left side chest area multi-directional opening. The right side chest area multi-directional opening includes a first substantially vertical disengagement area and a first substantially horizontal disengagement area. The left side chest area multi-directional opening includes a second substantially vertical disengagement area and a second substantially horizontal disengagement area.

### PRIORITY INFORMATION

The present application claims priority, under 35 U.S.C. § 119(e), from U.S. Provisional patent application, Ser. No. 62/553,277, filed on Sep. 1, 2017. The entire content of U.S. Provisional patent application, Ser. No. 62/553,277, filed on Sep. 1, 2017, is hereby incorporated by reference.

### BACKGROUND

Conventional garments for use with medical services or procedures have utilized various configurations to allow medical personnel access to medical devices and systems interacting with the patient.

For example, as illustrated in FIG. 1, a garment 10 includes an access opening 15 on an arm of the garment 10. The access opening 15 consists of a single slit that is closed or opened via a zipper mechanism, a latch and hook system, a plurality of snaps, or other system that allows the slit to be opened for access to the patient's arm and any medical devices attached to the patient's arm, such as an intravenous device.

Another example of a conventional garment for use with medical services or procedures is illustrated in FIG. 2. As illustrated in FIG. 2, a conventional garment 10 includes access openings 15 on the arms of the garment 10 and access openings 20 in the chest (pectoral) area of the garment 10.

The access openings (15 and 20) consist of single slits that are closed or opened via a zipper mechanism, a latch and hook system, a plurality of snaps, or other system that allows the slits to be opened for access to the patient's body (arm or torso) and any medical devices attached to the patient's body (arm or torso), such as an intravenous device or catheter.

As illustrated in FIG. 2, the various access openings (15 and 20) provide a unidirectional opening. More specifically, the disengagement of one garment portion attached to one side of the zipper mechanism, a latch and hook system, or plurality of snaps from another garment portion attached to the other side of the zipper mechanism, a latch and hook system, or plurality of snaps is in one direction, thereby forming a slit-like access opening.

In the conventional garments, described above with respect to FIG. 2, the slit-like access opening 20 in the chest area may not adequately close during a medical procedure, thereby leaving the patient's chest area exposed in a non-modest manner.

Furthermore, in the conventional garments, described above with respect to FIG. 2, the slit-like access opening 20 in the chest area provides a unidirectional opening which may not have a large enough opening to facilitate the medical service or procedure being provided to the patient.

Moreover, in the conventional garments, described above with respect to FIG. 2, the slit-like access opening 20 in the chest area fails to provide management of any tubes (mechanism to secure the tubes from movement) that may being used during the medical services or procedures. By failing to constrain the medical tubes, movement by the tubes may cause pain or discomfort to the patient and/or cause the tubes (needles attached to the patient's body) to disengage.

Therefore, it is desirable to provide an access opening in the chest area of a garment which can be properly closed during a medical procedure, thereby leaving the patient's chest area unexposed, thereby avoiding a non-modest situation.

Moreover, it is desirable to provide an access opening in the chest area of a garment which is non-slit like (multi-directional opening), thereby providing a large enough opening to facilitate the medical service or procedure being provided to the patient.

Furthermore, it is desirable to provide an access opening in the chest area of a garment that constrains the tubes being utilized in the medical service or procedure being provided to the patient.

In addition, it is desirable to provide an access opening in the chest area of a garment that can be properly closed during a medical procedure, is non-slit like (multi-directional opening), and constrains the tubes being utilized in the medical service or procedure being provided to the patient.

### BRIEF DESCRIPTION OF THE DRAWINGS

The drawings are only for purposes of illustrating various embodiments and are not to be construed as limiting, wherein:

FIG. 1 illustrates a conventional garment;

FIG. 2 illustrates another conventional garment;

FIG. 3 illustrates a garment with a chest area multi-directional opening, in a closed state, and a shoulder area tube management system;

FIG. 4 illustrates a garment with a chest area multi-directional opening, in an opened state, and a shoulder area tube management system;

FIG. 5 illustrates a tube management system associated with a chest area multi-directional opening;

FIG. 6 illustrates a tube management system associated with a shoulder area of the garment;

FIG. 7 illustrates another garment with a chest area multi-directional opening, in a closed state, a shoulder area tube management system, and a sleeve opening; and

FIG. 8 illustrates a garment with a chest area multi-directional opening, in an opened state, a shoulder area tube management system, and a sleeve opening.

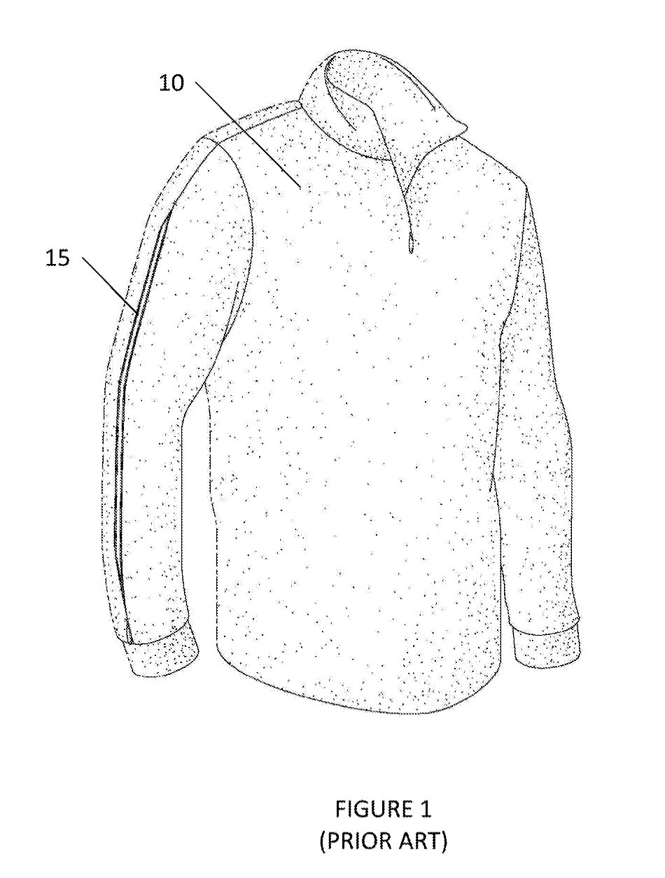

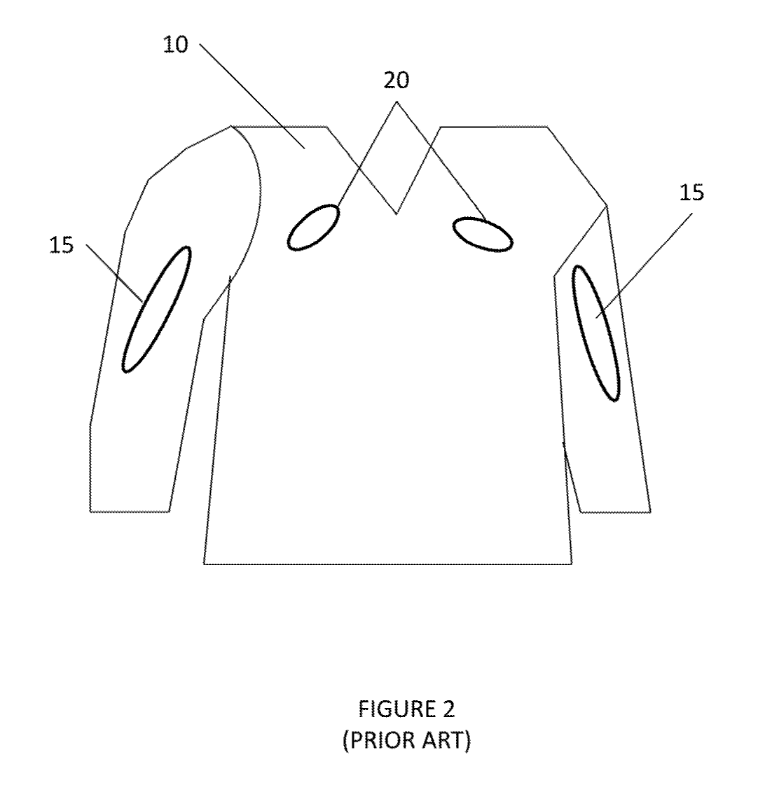

### DETAILED DESCRIPTION

For a general understanding, reference is made to the drawings. In the drawings, like references have been used throughout to designate identical or equivalent elements. It is also noted that the drawings may not have been drawn to scale and that certain regions may have been purposely drawn disproportionately so that the features and concepts may be properly illustrated.

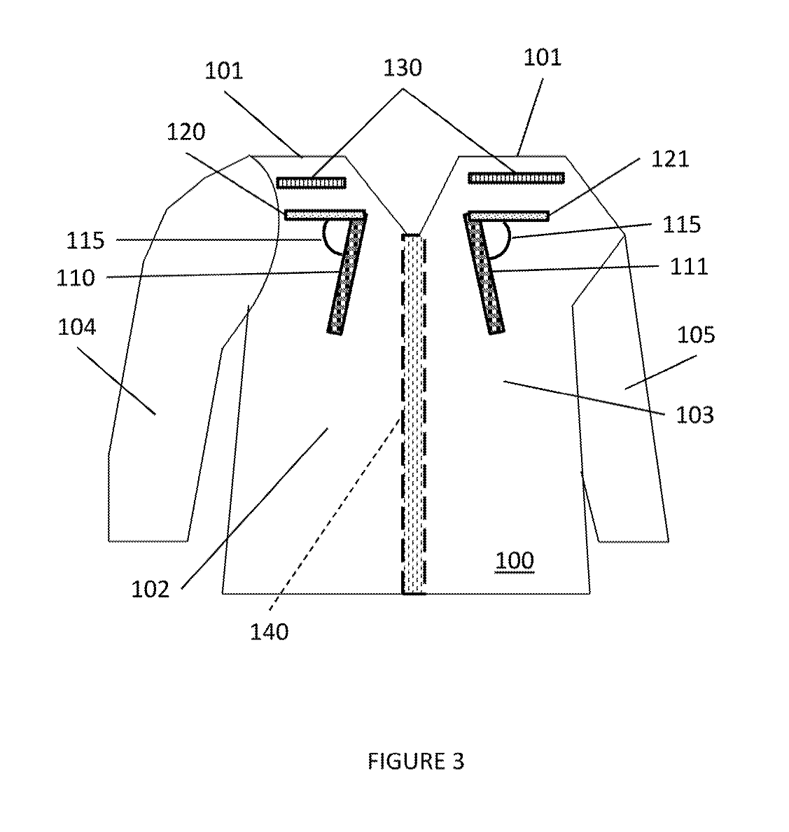

FIG. 3 illustrates a garment 100 with a right side chest area multi-directional opening (110 and 120), in a closed state, a left side chest area multi-directional opening (111 and 121), in a closed state, and a shoulder area tube management system 130. The garment 100 may include a zipper system 140 to allow the garment 100 to open up for easy on and off and close up to substantially seal the volume within the garment from the ambient environment.

The garment 100 includes a shoulder area 101, a right sleeve 104, a left sleeve 105, a right torso section 102, and a left torso section 103.

As illustrated in FIG. 3, the right side chest area multi-directional opening (110 and 120) is located in an upper portion of the right torso section 102 and includes the substantially vertical disengagement area 110 and the substantially horizontal disengagement area 120.

The left side chest area multi-directional opening (111 and 121) is located in an upper portion of the left torso section 103 and includes the substantially vertical disengagement area 110 and the substantially horizontal disengagement area 120.

It is noted that the angles 115 between the substantially vertical disengagement areas (110 and 111) and the substantially horizontal disengagement areas (120 and 121) are preferably between 45° and 90°.

The substantially vertical disengagement areas (110 and 111) utilize a zipper mechanism, a latch and hook system, or plurality of snaps to enable the disengagement of one portion of the garment from another portion of the garment in a substantially vertical direction.

The substantially horizontal disengagement areas (120 and 121) utilize a latch and hook system or similar system which can provide multiple independent non-continuous engagement sites to enable the disengagement of one portion of the garment from another portion of the garment in a substantially horizontal direction.

With respect to providing multiple independent non-continuous engagement sites, a latch and hook system can be engaged anywhere along the system such that one engagement site need not be continuous or contiguous to another engagement site.

Moreover, with respect to providing multiple independent non-continuous engagement sites, a latch and hook system can be engaged to provide multiple independent access openings along the latch and hook system.

Lastly, with respect to providing multiple independent non-continuous engagement sites, a latch and hook system can be engaged to provide multiple independent access openings along the latch and hook system which enables the individual securing of a medical tube or a medical wire within the independent access opening.

In contrast, although a zipper mechanism has multiple engagement sites, the engagement is continuous and dependent upon the engagement of a previously engaged engagement site.

In other words, a zipper mechanism provides a single continuous access opening. The zipper mechanism cannot provide multiple independent non-continuous engagement sites, thereby cannot provide multiple independent access openings along the zipper mechanism.

More specifically, the zipper mechanism cannot provide the individual securing of a medical tube or a medical wire within the independent access opening.

The multiple independent access openings are more clearly illustrated in FIG. 5.

As illustrated in FIG. 5, the substantially horizontal disengagement area 1200 includes a first engagement mechanism 1240 including either latches or hooks and a second engagement mechanism 1220 including either latches or hooks. If the first engagement mechanism 1240 includes latches, the second engagement mechanism 1220 includes hooks.

On the other hand, if the first engagement mechanism 1240 includes hooks, the second engagement mechanism 1220 includes latches.

The latch and hook system of the substantially horizontal disengagement area 1200 of FIG. 5 can be engaged at multiple engagement sites 1250 (where the hooks engage the latches to form a detachable bond therebetween). The various engagement sites 1250 can be positioned, as illustrated, so as to form discreet hollow volumes or cylinders 1280 so that tubes or other medical devices (wires) 1500 can run through the discreet hollow volumes or cylinders 1280.

Moreover, since the latch and hook system of the substantially horizontal disengagement area 1200 of FIG. 5 can be engaged anywhere along the system, the size of the discreet hollow volumes or cylinders 1280 can be dynamic (the cross area or diameter of the hollow volume or cylinder can be changed) so that the first engagement mechanism 1240 can securely fit around the tubes or other medical devices (wires) 1500, thereby preventing undesirable movement or shifting of the tubes or other medical devices (wires) 1500.

It is further noted that the substantially vertical disengagement area 1100 and substantially horizontal disengagement area 1200 can be opened and closed independently of each other, thereby providing modestly for the patient.

With respect to the shoulder area tube management system 1300, the shoulder area tube management system 1300 can utilize a latch and hook system or similar system (as illustrated in FIG. 5) which can provide multiple independent non-continuous engagement sites to enable the disengagement of one portion of the garment from another portion of the garment.

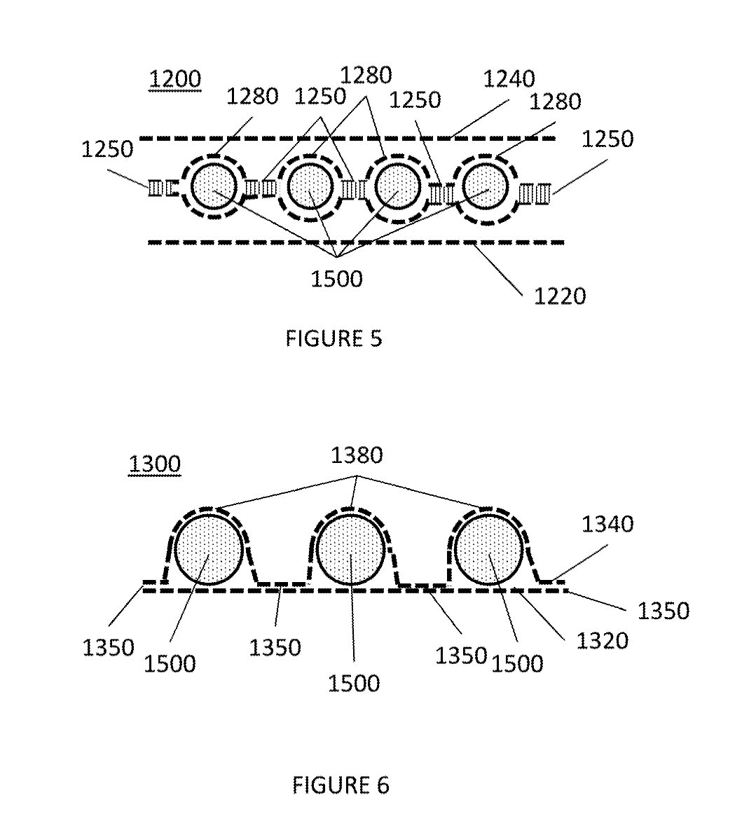

Alternatively, as illustrated in FIG. 6, the shoulder area tube management system 1300 can utilize a fabric strap 1340 which is detachably attached (substantially permanent) to the shoulder area 1320 of the garment 100 at multiple locations to provide multiple independent attachment sites 1350, thereby providing discreet hollow volumes or cylinders 1380.

The discreet hollow volumes or cylinders 1380 provide a channel or path for tubes or other medical devices (wires) 1500 to run therethrough. Since the fabric strap 1340 is detachably attached (substantially permanent) to the shoulder area 1320 of the garment 100, the size of the discreet hollow volumes or cylinders 1280 is static.

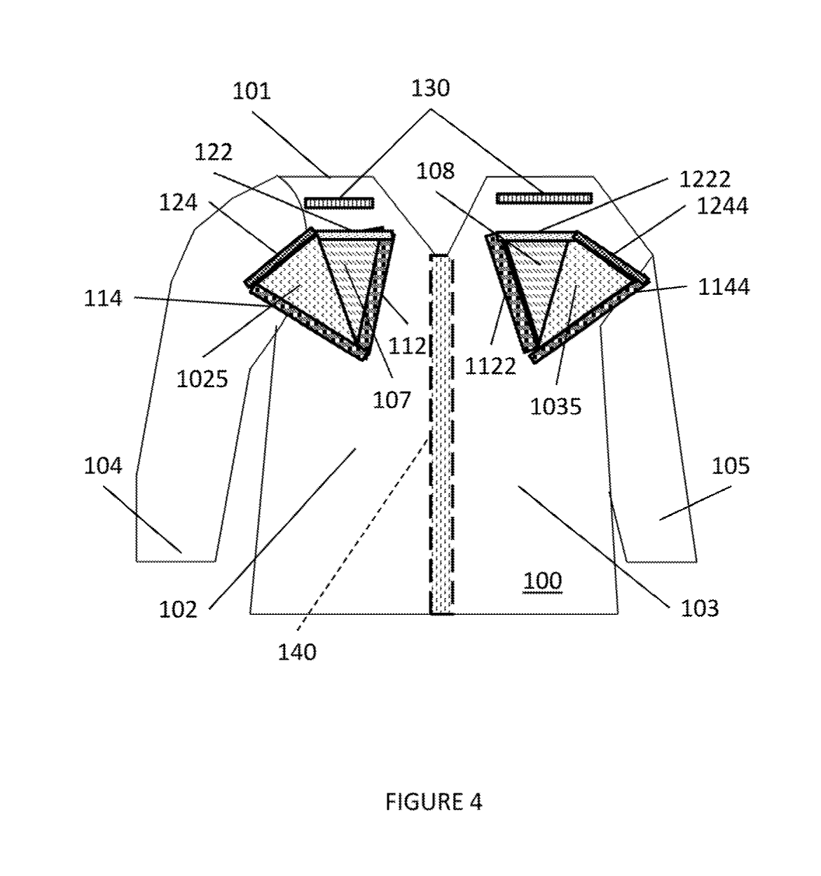

FIG. 4 illustrates a garment with the right side chest area multi-directional opening, in an opened state, the left side chest area multi-directional opening, in an opened state, and a shoulder area tube management system 130.

As illustrated in FIG. 4, with respect to the right side chest area multi-directional opening in the opened state, the first engagement mechanism 124 of the substantially horizontal disengagement area is disengaged from the second engagement mechanism 122 of the substantially horizontal disengagement area. In addition, with respect to the right side chest area multi-directional opening in the opened state, a first engagement mechanism 112 of the substantially vertical disengagement area is disengaged from a second engagement mechanism 114 of the substantially vertical disengagement area.

With respect to the left side chest area multi-directional opening in the opened state, the first engagement mechanism 1244 of the substantially horizontal disengagement area is disengaged from the second engagement mechanism 1222 of the substantially horizontal disengagement area. In addition, with respect to the left side chest area multi-directional opening in the opened state, a first engagement mechanism 1122 of the substantially vertical disengagement area is disengaged from a second engagement mechanism 1144 of the substantially vertical disengagement area.

As further illustrated in FIG. 4, with respect to the right side chest area multi-directional opening in the opened state, when to the right side chest area multi-directional opening is opened, the back panel 107 of the garment 100 is exposed, and the backside 1025 of the right torso section 102 of the garment 100 is exposed.

As additionally illustrated in FIG. 4, with respect to the left side chest area multi-directional opening in the opened state, when to the left side chest area multi-directional opening is opened, the back panel 108 of the garment 100 is exposed, and the backside 1035 of the right torso section 103 of the garment 100 is exposed.

It is noted that although the illustration of FIG. 4 shows an opening from a center of the garment to an outer portion of the garment, the access are could be configured to open from an outer portion of the garment to a center of the garment.

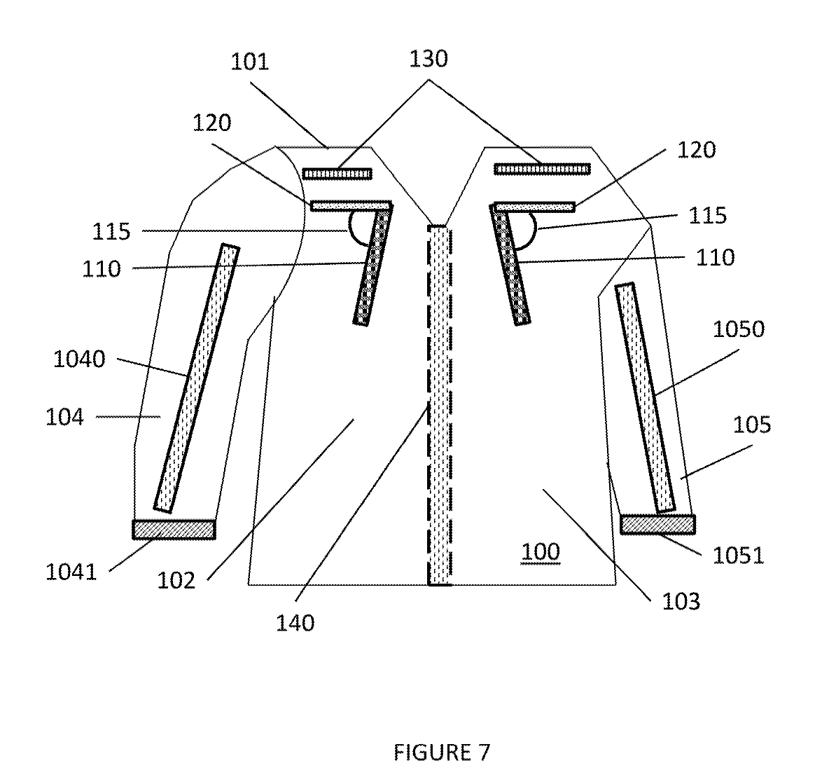

FIG. 7 illustrates a garment 100 with a right side chest area multi-directional opening (110 and 120), in a closed state, a left side chest area multi-directional opening (111 and 121), in a closed state, and a shoulder area tube management system 130. The garment 100 may include a zipper system 140 to allow the garment 100 to open up for easy on and off and closing to substantially seal the volume within the garment from the ambient environment.

The garment 100 includes a shoulder area 101, a right sleeve 104, a left sleeve 105, a right torso section 102, and a left torso section 103.

As illustrated in FIG. 7, the right side chest area multi-directional opening (110 and 120) is located in an upper portion of the right torso section 102 and includes the substantially vertical disengagement area 110 and the substantially horizontal disengagement area 120.

The left side chest area multi-directional opening (111 and 121) is located in an upper portion of the left torso section 103 and includes the substantially vertical disengagement area 110 and the substantially horizontal disengagement area 120.

It is noted that the angles 115 between the substantially vertical disengagement areas (110 and 111) and the substantially horizontal disengagement areas (120 and 121) are preferably between 45° and 90°.

As illustrated in FIG. 7, the right sleeve 104 may include a sleeve opening 1040, which utilize a zipper mechanism, a latch and hook system, or plurality of snaps to enable the disengagement of one portion of the garment from another portion of the garment in a substantially vertical direction.

The right sleeve 104 may further include a cuff 1041 to ensure a snug fit of the end of the right sleeve 104 with a limb of a user.

The cuff 1041 may also include a disengagement mechanism, such as a snap, to enable an opening of the cuff 1041 and the falling away of the right sleeve 104 from the limb of the user.

As illustrated in FIG. 7, the left sleeve 105 may include a sleeve opening 1050, which utilize a zipper mechanism, a latch and hook system, or plurality of snaps to enable the disengagement of one portion of the garment from another portion of the garment in a substantially vertical direction.

The left sleeve 105 may further include a cuff 1051 to ensure a snug fit of the end of the left sleeve 105 with a limb of a user.

The cuff 1051 may also include a disengagement mechanism, such as a snap, to enable an opening of the cuff 1051 and the falling away of the left sleeve 105 from the limb of the user.

The substantially vertical disengagement areas (110 and 111) utilize a zipper mechanism, a latch and hook system, or plurality of snaps to enable the disengagement of one portion of the garment from another portion of the garment in a substantially vertical direction.

The substantially horizontal disengagement areas (120 and 121) utilize a latch and hook system or similar system which can provide multiple independent non-continuous engagement sites to enable the disengagement of one portion of the garment from another portion of the garment in a substantially horizontal direction.

With respect to providing multiple independent non-continuous engagement sites, a latch and hook system can be engaged anywhere along the system such that one engagement site need not be continuous or contiguous to another engagement site.

Moreover, with respect to providing multiple independent non-continuous engagement sites, a latch and hook system can be engaged to provide multiple independent access openings along the latch and hook system.

Lastly, with respect to providing multiple independent non-continuous engagement sites, a latch and hook system can be engaged to provide multiple independent access openings along the latch and hook system which enables the individual securing of a medical tube or a medical wire within the independent access opening.

In contrast, although a zipper mechanism has multiple engagement sites, the engagement is continuous and dependent upon the engagement of a previously engaged engagement site.

In other words, a zipper mechanism provides a single continuous access opening. The zipper mechanism cannot provide multiple independent non-continuous engagement sites, thereby cannot provide multiple independent access openings along the zipper mechanism.

More specifically, the zipper mechanism cannot provide the individual securing of a medical tube or a medical wire within the independent access opening.

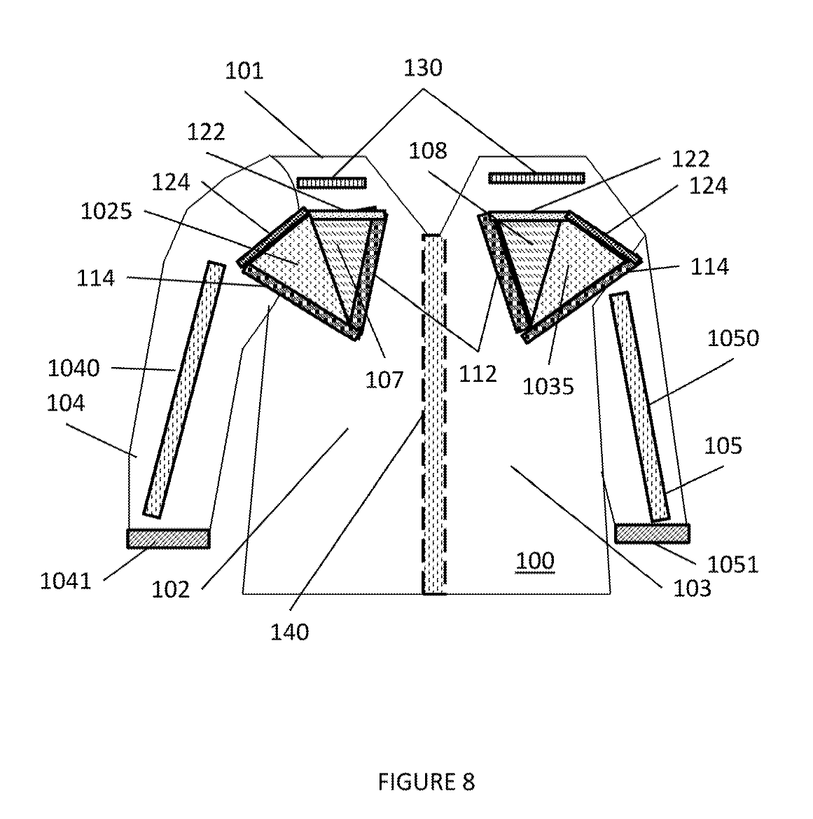

FIG. 8 illustrates a garment with the right side chest area multi-directional opening, in an opened state, the left side chest area multi-directional opening, in an opened state, and a shoulder area tube management system 130.

As illustrated in FIG. 8, with respect to the right side chest area multi-directional opening in the opened state, the first engagement mechanism 124 of the substantially horizontal disengagement area is disengaged from the second engagement mechanism 122 of the substantially horizontal disengagement area. In addition, with respect to the right side chest area multi-directional opening in the opened state, a first engagement mechanism 112 of the substantially vertical disengagement area is disengaged from a second engagement mechanism 114 of the substantially vertical disengagement area.

With respect to the left side chest area multi-directional opening in the opened state, the first engagement mechanism 1244 of the substantially horizontal disengagement area is disengaged from the second engagement mechanism 1222 of the substantially horizontal disengagement area. In addition, with respect to the left side chest area multi-directional opening in the opened state, a first engagement mechanism 1122 of the substantially vertical disengagement area is disengaged from a second engagement mechanism 1144 of the substantially vertical disengagement area.

As further illustrated in FIG. 8, with respect to the right side chest area multi-directional opening in the opened state, when to the right side chest area multi-directional opening is opened, the back panel 107 of the garment 100 is exposed, and the backside 1025 of the right torso section 102 of the garment 100 is exposed.

As additionally illustrated in FIG. 8, with respect to the left side chest area multi-directional opening in the opened state, when to the left side chest area multi-directional opening is opened, the back panel 108 of the garment 100 is exposed, and the backside 1035 of the right torso section 103 of the garment 100 is exposed.

As illustrated in FIG. 8, the right sleeve 104 may include a sleeve opening 1040, which utilize a zipper mechanism, a latch and hook system, or plurality of snaps to enable the disengagement of one portion of the garment from another portion of the garment in a substantially vertical direction.

The right sleeve 104 may further include a cuff 1041 to ensure a snug fit of the end of the right sleeve 104 with a limb of a user.

The cuff 1041 may also include a disengagement mechanism, such as a snap, to enable an opening of the cuff 1041 and the falling away of the right sleeve 104 from the limb of the user.

As illustrated in FIG. 8, the left sleeve 105 may include a sleeve opening 1050, which utilize a zipper mechanism, a latch and hook system, or plurality of snaps to enable the disengagement of one portion of the garment from another portion of the garment in a substantially vertical direction.

The left sleeve 105 may further include a cuff 1051 to ensure a snug fit of the end of the left sleeve 105 with a limb of a user.

The cuff 1051 may also include a disengagement mechanism, such as a snap, to enable an opening of the cuff 1051 and the falling away of the left sleeve 105 from the limb of the user.

It is noted that although the illustration of FIG. 8 shows an opening from a center of the garment to an outer portion of the garment, the access are could be configured to open from an outer portion of the garment to a center of the garment.

In another embodiment, the garment includes a substantially horizontal disengagement area and a substantially vertical disengagement area located in the chest region of the garment.

The substantially horizontal disengagement area may include a latch and hook system for enabling the opening and closing of the substantially horizontal disengagement area and to enable the creation of hollow volumes or cylinders to secure tubes or other medical devices. The substantially vertical disengagement area also may include a latch and hook system for enabling the opening and closing of the substantially vertical disengagement area. The substantially vertical disengagement area may further include a zipper mechanism for enabling the opening and closing of the substantially vertical disengagement area. The substantially vertical disengagement area may additionally include a plurality of snaps for enabling the opening and closing of the substantially vertical disengagement area. The substantially horizontal disengagement area may include an attachment system which can provide multiple independent non-continuous engagement sites to enable the disengagement of one portion of the garment from another portion of the garment and to enable the creation of hollow volumes or cylinders to secure tubes or other medical devices.

In a further embodiment, the garment includes a tube management system located in the shoulder region of the garment.

The tube management system may include a latch and hook system for enabling the securing of tubes or medical devices in the tube management system by creating hollow volumes or cylinders to secure tubes or other medical devices.

The tube management system may include a plurality of snaps for enabling the securing of tubes or medical devices in the tube management system by creating hollow volumes or cylinders to secure tubes or other medical devices.

The tube management system may include a fabric strap that is detachably attached to the shoulder region of the garment for enabling the securing of tubes or medical devices in the tube management system by creating hollow volumes or cylinders to secure tubes or other medical devices.

An additional embodiment of the garment includes a substantially horizontal disengagement area, a substantially vertical disengagement area located in the chest region of the garment, and a tube management system located in the shoulder region of the garment.

It is noted that the garment may be a pullover, thereby not requiring the zipper system.

It is further noted that in the various zipper systems described above, the zipper system may be unidirectional wherein a single zipper moves in one direction to create the opening and moves in the opposite direction to close the opening, or the zipper system may be bi-directional wherein two zippers move in opposite directions to create the opening and the zippers move in the corresponding opposite directions to close the opening.

In summary, a garment comprises a right side chest area multi-directional opening and a left side chest area multi-directional opening; the right side chest area multi-directional opening including a first substantially vertical disengagement area and a first substantially horizontal disengagement area; the left side chest area multi-directional opening including a second substantially vertical disengagement area and a second substantially horizontal disengagement area.

The first substantially vertical disengagement area of the right side chest area multi-directional opening may include a first zipper system; the second substantially vertical disengagement area of the left side chest area multi-directional opening may include a second zipper system; the first substantially vertical disengagement area of the right side chest area multi-directional opening may include a first latch and hook system; the second substantially vertical disengagement area of the left side chest area multi-directional opening may include a second latch and hook system; the first substantially vertical disengagement area of the right side chest area multi-directional opening may include a first plurality of snaps; and the second substantially vertical disengagement area of the left side chest area multi-directional opening may include a second plurality of snaps.

The first substantially horizontal disengagement area of the right side chest area multi-directional opening may include a first latch and hook system; the second substantially horizontal disengagement area of the left side chest area multi-directional opening may include a second latch and hook system; the first substantially horizontal disengagement area of the right side chest area multi-directional opening may include a plurality of snaps; and the second substantially horizontal disengagement area of the left side chest area multi-directional opening may include a second plurality of snaps.

The garment may include a shoulder area tube management system. The shoulder area tube management system may include multiple independent non-continuous engagement sites to enable disengagement of one portion of the garment from another portion of the garment. The shoulder area tube management system may include a latch and hook system. The shoulder area tube management system may include a plurality of snaps.

The may include a first sleeve and a second sleeve; the first sleeve including a first zipper system to enable an opening in the first sleeve; the second sleeve including a second zipper system to enable an opening in the second sleeve.

It will be appreciated that several of the above-disclosed embodiments and other features and functions, or alternatives thereof, may be desirably combined into many other different systems or applications. Also, various presently unforeseen or unanticipated alternatives, modifications, variations, or improvements therein may be subsequently made by those skilled in the art which are also intended to be encompassed by the description above.

What is claimed is:

1. A garment comprising:
a right side chest area multi-directional opening; and
a left side chest area multi-directional opening;
said right side chest area multi-directional opening including,
a first substantially vertical disengagement area, and
a first substantially horizontal disengagement area;
said left side chest area multi-directional opening including,
a second substantially vertical disengagement area, and
a second substantially horizontal disengagement area.

2. The garment as claimed in claim 1, wherein said first substantially vertical disengagement area of said right side chest area multi-directional opening includes a first zipper system;
said second substantially vertical disengagement area of said left side chest area multi-directional opening includes a second zipper system.

3. The garment as claimed in claim 1, wherein said first substantially vertical disengagement area of said right side chest area multi-directional opening includes a first latch and hook system;
said second substantially vertical disengagement area of said left side chest area multi-directional opening includes a second latch and hook system.

4. The garment as claimed in claim 1, wherein said first substantially vertical disengagement area of said right side chest area multi-directional opening includes a first plurality of snaps;
said second substantially vertical disengagement area of said left side chest area multi-directional opening includes a second plurality of snaps.

5. The garment as claimed in claim 1, wherein said first substantially horizontal disengagement area of said right side chest area multi-directional opening includes a first latch and hook system;
said second substantially horizontal disengagement area of said left side chest area multi-directional opening includes a second latch and hook system.

6. The garment as claimed in claim 2, wherein said first substantially horizontal disengagement area of said right side chest area multi-directional opening includes a first latch and hook system;
said second substantially horizontal disengagement area of said left side chest area multi-directional opening includes a second latch and hook system.

7. The garment as claimed in claim 3, wherein said first substantially horizontal disengagement area of said right side chest area multi-directional opening includes a first latch and hook system;
said second substantially horizontal disengagement area of said left side chest area multi-directional opening includes a second latch and hook system.

8. The garment as claimed in claim 4, wherein said first substantially horizontal disengagement area of said right side chest area multi-directional opening includes a first latch and hook system;
said second substantially horizontal disengagement area of said left side chest area multi-directional opening includes a second latch and hook system.

9. The garment as claimed in claim 1, wherein said first substantially horizontal disengagement area of said right side chest area multi-directional opening includes a plurality of snaps;
said second substantially horizontal disengagement area of said left side chest area multi-directional opening includes a second plurality of snaps.

10. The garment as claimed in claim 2, wherein said first substantially horizontal disengagement area of said right side chest area multi-directional opening includes a first plurality of snaps;
said second substantially horizontal disengagement area of said left side chest area multi-directional opening includes a second plurality of snaps.

11. The garment as claimed in claim 3, wherein said first substantially horizontal disengagement area of said right side chest area multi-directional opening includes a first plurality of snaps;
said second substantially horizontal disengagement area of said left side chest area multi-directional opening includes a second plurality of snaps.

12. The garment as claimed in claim 4, wherein said first substantially horizontal disengagement area of said right side chest area multi-directional opening includes a first plurality of snaps;
said second substantially horizontal disengagement area of said left side chest area multi-directional opening includes a second plurality of snaps.

13. The garment as claimed in claim 1, further comprising a shoulder area tube management system.

14. The garment as claimed in claim 13, wherein said shoulder area tube management system includes multiple independent non-continuous engagement sites to enable disengagement of one portion of the garment from another portion of the garment.

15. The garment as claimed in claim 14, wherein said shoulder area tube management system includes a latch and hook system.

16. The garment as claimed in claim 14, wherein said shoulder area tube management system includes a plurality of snaps.

17. The garment as claimed in claim 1, further comprising:
a first sleeve; and
a second sleeve;
said first sleeve including a first zipper system to enable an opening in said first sleeve;
said second sleeve including a second zipper system to enable an opening in said second sleeve.
